(19) United States
(12) Patent Application Publication
(10) Pub. No.: US 2015/0225770 A1
(43) Pub. Date: Aug. 13, 2015

(54) PHENOTYPE-NEUTRAL BARCODES FOR DIGITAL ANALYSIS

(71) Applicant: U.S. Army Research Laboratory ATTN: RDRL-LOC-I, Adelphi, MD (US)

(72) Inventors: Dontcho V. Jelev, Rockville, MD (US); Henry S. Gibbons, Halethorpe, MD (US); Tsvetanka S. Zheleva, Rockville, MD (US)

(73) Assignee: U.S. Army Research Laboratory ATTN: RDRL-LOC-I, Adelphi, MD (US)

(21) Appl. No.: 14/178,665

(22) Filed: Feb. 12, 2014

(51) Int. Cl. C12Q 1/68

(52) U.S. Cl. CPC C12Q 1/6806; C12Q 1/6888; C12Q 2600/16; C12Q 2600/156

(57) ABSTRACT

Processes of specifically and effectively labeling an organism are provided. Processes involve the incorporation of a plurality of phenotype neutral tags that are differentially detected where the presence or absence of the tag is represented by a digital readout. The incorporation of stealth tags or insertion tags provides a rapid and population maintaining labeling of an organism that can be readily identified by digital PCR techniques.

### GOVERNMENT INTEREST

The invention described herein may be manufactured, used, and licensed by or for the United States Government.

### FIELD OF THE INVENTION

The invention relates to identification of organisms by genetic tagging and digital analysis. More specifically, the invention relates to phenotype-neutral tagging of organisms and their rapid digital detection in a background of untagged or environmentally derived materials. A genetic barcoding is provided that allows rapid recognition of a tagged organism by a digital readout representative of the specifically tagged organism.

### BACKGROUND OF THE INVENTION

Significant scientific, biotechnology, and medical interest exists for determining the fate of cellular types in mixed populations. Gaining a better understanding how organisms in a population are effected by environmental changes or human intervention has many applications including tracing the effects of pesticides, tracking of live vaccines, water quality control, and better understanding population dynamics of microorganisms in the environment.

Studying the environmental fate of bacteria is hampered by a limited number of available phenotype neutral tracer strains not carrying drug resistance genes. For example, during the last 70 years the U.S. Army has used two to three strains of B. atrophaeus var globigii (BG) as simulants for B. anthracic. This has resulted in building high levels of background at testing grounds. Understanding the fate of microorganisms or monitoring eradication of these organisms requires some form of tagging to discern a known strain from an environmentally introduced strain.

One method of tagging is through the use of genomic labels. Genomic labeling processes are commonly referred to as genetic footprinting where unique DNA sequence tags serve as molecular “barcode” identifiers for specific variants in a mixed population. Present day genomic barcodes use two tags. This design stems from the classical barcoded deletion mutants used in functional genomics. Deletion mutants render genes non-functional and amenable to insertion of other sequences such as drug resistances for strain selection. Deletion mutants use thousands of unique designer tags inserted at sites of deletion including the introduction of an antibiotic resistance gene or other identifiable tag. However, this design is not easily extendable for use with phenotype-neutral barcodes where barcodes are expected to have minimum effect of the host fitness. In addition, presently used barcode designs do not allow the use of next generation digital analysis platforms such as digital PCR to their full potential. This limits the simultaneously used barcoded strains in mixed populations to the number of available channels of the detecting PCR instrument.

As such, new methods for the tagging and identification of source or target organisms are needed.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

Provided are processes of labeling an organism, and optionally of later detecting an organism in a sample such as an environmental sample. The invention incorporates a plurality of tags into the genome of an organism that are detectable as a digital barcode specific to the organism labeled and its progeny. The processes provide for highly specific recognition of a labeled organism in a population of like or dislike background organisms.

A process for labeling an organism is provided. Some embodiments of a process include selecting a labeling region within an exon of a gene of the organism and substituting a plurality of non-wild type nucleotides within the labeling region to produce a plurality of tags within the labeling region, the plurality of tags representing a barcode, at least two of the plurality of tags each in a separate codon resulting in synonymous substitutions at each nucleotide substitution site thereby producing silent mutations in the gene such that a protein encoded by the gene is the wild-type amino acid sequence. A labeling region preferably includes two primer binding sites flanking the plurality of tags where the primer binding sites are a pair suitable for amplification of the labeling region by a single pair of primers, each binding to one of the primer binding sites, to produce an amplification product. The creation of barcodes in the exon of a gene of an organism optionally incorporates four non-wild type nucleotides with at least one in each of four tags. A tag may include more than one non-wild type nucleotide.

In some embodiments the process further includes amplifying the entire plurality of tags by hybridizing a forward primer to the labeling region at a first primer binding site, and hybridizing a reverse primer to the labeling region at a second primer binding site; and subjecting the labeling region to PCR amplification producing an amplification product comprising all of the tags. A plurality of probes is optionally contacted to the amplification product and the presence or absence of hybridization of each of the probes is detected. Detection is optionally by real-time PCR methods, mass spectrometry methods, or other method known in the art for detecting hybridization of a probe to a target sequence.

The gene is preferably an essential, coessential, conditionally essential, or phenotype supporting gene. The presence of the tags in some embodiments of the process does not alter the phenotype of the target organism, however. The introduction of tags into the exon of a gene is performed by creating synonymous substitutions within the gene that are detectable on the genetic level only. The organism will not respond to the tag as the protein produced by the gene following transcription and translation will be identical between a tagged organism and a non-tagged organism.

An alternative process of labeling an organism is also provided that includes selecting an insertion site in a non-coding region of a genome of an organism, inserting at the insertion site a plurality of nucleotides within the non-coding region, optionally by markerless recombination, to produce an insertion sequence comprising a plurality of tags, where the plurality of tags constitute a barcode or portion thereof; and optionally amplifying the insertion sequence by a polymerase chain reaction using a single pair of primers comprising a forward primer and a reverse primer to produce an amplification product. The insertion sequence is not recognized by the restriction modification system of the target organism. Also, the insertion sequence does not introduce genetic material or delete genetic material that alters the phenotype of the tagged organism relative to a like non-tagged organism. In these embodiments, a non-coding region is optionally 500 nucleotides or larger. The non-coding region is optionally flanked by convergently transcribed genes. In some embodiments, the non-coding region is free from identical repetitive elements of 200 nucleotides or more within 10,000 nucleotides from the insertion site. Optionally a process further includes contacting a plurality of probes with the amplification product and detecting the presence or absence of hybridization of each of the probes to a corresponding tag. Optionally, the presence or absence of hybridization of a probe to a tag is used to generate a barcode that is read or readable as a digital output. Optionally, a digital output is created such as by a computer specially configured to produce such a digital output.

Also provided are processes of identifying an organism in a sample. A process includes producing an amplification product by amplifying a nucleotide sequence comprising two or more tags, at least two of the tags being non-wild type in nucleotide sequence, using a forward primer that hybridizes to a first region within a labeling region within a genome of the organism, and a reverse primer that hybridizes to a second region within the labeling region, under conditions suitable for a polymerase chain reaction; contacting the amplification product with a plurality of probes each specific for one of the tags; detecting the presence or absence of each of the tags in the amplification product by hybridization of one or more of the probes to the amplification product to produce a barcode readout; and identifying the organism by the constitution of the barcode readout. Optionally, a listing of barcoded organisms is used to compare the digital readout to the list of digital signatures to identify the organism. Optionally, four or more probes are contacted to the amplification product, optionally five or more probes are contacted to the amplification product. Each of the probes includes a unique label that is distinguishable from the other probes in the probe set. Optionally, the nucleotide sequence is entirely within a gene of the organism, yet the organism is phenotypically indistinguishable from a non-tagged like organism.

The processes provided allow for specific and sensitive monitoring of tagged organisms in a population without population effects due to the presence of the tagged organisms. Rapid and sensitive digital readouts allow for the labeling of a large number of similar or identical organisms and accurate following of each of the organisms in an environmental laboratory situation.

### BRIEF DESCRIPTION OF THE DRAWINGS

FIG. 1 illustrates a labeling region including four tags under the control of a single set of primers to produce a barcode as well as how each barcode results in a fluorescence readout of the presence or absence of hybridization of a probe to the tag and the digital readout of the barcode encoded by the presence or absence of the tags in the labeling region;

FIG. 2 illustrates module design of genomic barcodes using four tags and multiple primer sequences;

FIG. 3 illustrates the alignment of partial nucleic acid sequences from the Spo0A_C conserved domain of Spo0A as well as primer regions (lightly shaded) and tag locations (underlined); and

FIG. 4 illustrates an exemplary barcode set used for B. thuringiensis with the primer binding regions shown underlined, the tags in larger font text, and the tags that include a synonymous substitution in italics.

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the compositions are described as using specific materials in a particular order, it is appreciated that the described materials or order may be interchangeable such that the description of the invention includes multiple parts or steps arranged in many ways as is readily appreciated by one of skill in the art.

Methods for barcode design capable of use with modem instruments for digital analysis of nucleic acids such as digital PCR are provided. The invention has utility for the labeling and tracking of organisms in the laboratory or environment. The barcodes designed by the invention behave as digital information units. This allows a large number of barcodes belonging to a common barcode set to be placed in a single organism type. The barcodes are suitable for simultaneous detection of large number of barcoded strains when digital PCR instruments are used. This opens a unique opportunity for simultaneous use of multiple barcoded strains for studying population dynamics.

The invention is described as labeling an organism using a stealth barcode, an insertion barcode, or combinations thereof. Both stealth barcodes and insertion barcodes are phenotype neutral labeling methods.

“Phenotype neutral” is used herein to mean that the presence of a tag or plurality of tags does not alter the phenotype of the organism that is tagged. Stealth barcodes achieve phenotype neutrality in the presence of non-wild type sequences due to the introduction of synonymous mutations within a coding region of a target sequence thereby preserving the sequence of a protein encoded by the gene. As such, the resulting tagged organism is tagged at the genetic level alone and does not suffer fitness deficits or enhancements. An insertion barcode maintains phenotype neutrality by inserting the insertion sequence in a non-coding region of the organism's genome whereby the non-coding region does not produce a product or itself alter any phenotype characteristic of the organism. It will be appreciated that many descriptions of stealth barcodes and insertion barcodes will overlap as is recognized by one of ordinary skill in the art.

A process of labeling an organism with a plurality of tags that may be compiled in digital output format are provided. A process with respect to stealth barcoding includes selecting a labeling region within an exon of a gene. The labeling region is tagged by substituting a plurality of non-wild type nucleotides within the labeling region to produce a plurality of tags with at least two tags including at least one non-wild type nucleotide and at least two of the tags present in separate codons. Each non-wild-type nucleotide represents a synonymous substitution whereby the resulting codon encodes the same amino acid as the wild-type codon such that a protein encoded by the gene is the wild-type amino acid sequence.

A tag may or may not include a non-wild type nucleotide that represents a synonymous mutation within the gene that is tagged. The genetic code is degenerate meaning that more than one codon sequence will encode for incorporation of a single amino acid. For example, both the codons GAA and GAG specify glutamic acid. As such, and merely as a single illustration, substituting G at position 3 in the codon GAA will introduce a synonymous mutation in the gene that does not result in a phenotypic alteration in the organism due to the tagged gene encoding the identical protein as the wild-type gene sequence. A tag may include a section of nucleic acid sequence that has the wild-type sequence, but is chosen to work in concert with at least two tags that do include a codon with a synonymous mutation. As such, a tag represents a nucleic acid sequence within a labeling region that is either wild-type or includes one or more mutations. A tag includes from 5 to 30 nucleotides, optionally 5, 6, 7, 8, 9, 10, 11, 12, 13, 14, 15, 16, 17, 18, 19, 20, 21, 22, 23, 24, 25, 26, 27, 28, 29, or 30 nucleotides. Some embodiments include a tag that is longer in sequence. A tag is unique in nucleic acid sequence relative to all other tags in the organism such that each tag is differentially recognizable by a plurality of probes within the same sample volume as the labeling region.

The term “nucleotide” is intended to mean a base-sugar-phosphate combination either natural or synthetic. Included in this definition are modified nucleotides which include additions to the sugar-phosphate groups as well as to the bases. Natural nucleotides include adenine, guanine, thymine, cytosine, and uracil.

The term “nucleic acid” or “oligonucleotide” refers to multiple nucleotides attached in the form of a single or double stranded polynucleotide that can be natural, or derived synthetically, enzymatically, and by cloning methods. The term “oligonucleotide” refers to a polynucleotide of 2500 nucleotides or fewer, optionally 500 nucleotides or fewer, optionally 300 nucleotides or fewer, optionally 200 nucleotides or fewer. The terms “nucleic acid” and “oligonucleotide” may be used interchangeably in this application.

As used herein, the terms “subject” or “organism” are treated synonymously and are defined as any being that includes a gene, including a virus. A subject illustratively includes: a mammal including humans, non-human primates, horses, goats, cows, sheep, pigs, dogs, cats, and rodents; arthropods; single celled organisms illustratively bacteria; viruses; and cells.

An inventive process according to some embodiments includes substituting a plurality of non-wild type nucleotides within a labeling region to produce a plurality of tags within the labeling region. The number of non-wild type nucleotides is limited by the number of tags used, the tag length, and the maximum number of nucleotides substitutable to produce a synonymous mutation. A single tag may include more than one non-wild-type nucleotide. As a single non-limiting example, if a tag is 9 nucleotides long, three non-wild type nucleotides are optionally present that each produce a synonymous mutation in the coding sequence.

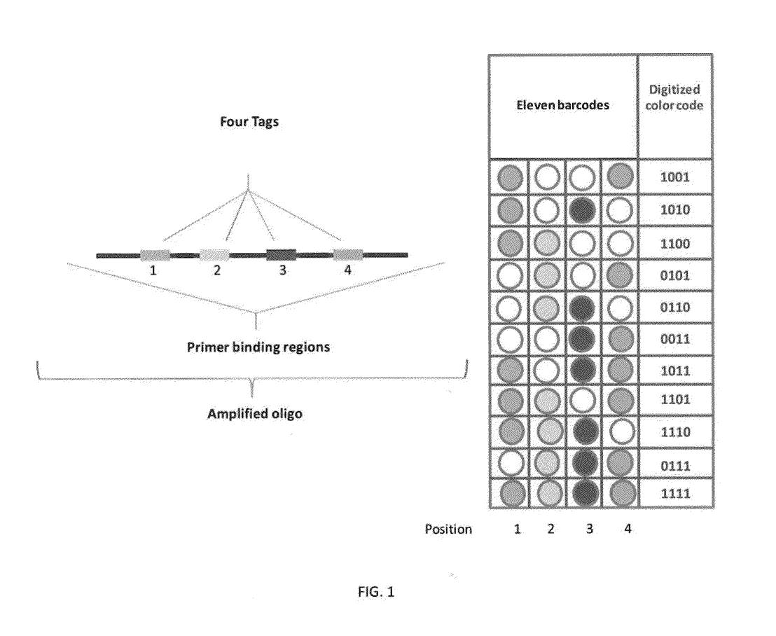

A plurality of tags is present in a labeling region. A plurality of tags represents a barcode with at least two of the tags including a non-wild type nucleotide. A barcode includes three or more tags. FIG. 1 illustrates an exemplary schema for barcode design that may be used in a four channel (color) digital PCR instrument. On the left the main elements of the design are shown. The labeling region carrying all tags is amplified using a single pair of primers. In this example, four regions available for tagging (tags) are chosen depicted as bars. Each tag is recognized by a dedicated probe labeled with a fluorescent (or other) label (e.g. FAM, HEX, ROX or Cy5). Upon amplification of the labeling region, the plurality of probes recognizing each of the tagged regions, are hybridized to the amplification product. The absence of hybridization of a probe to a tag is detected as below threshold fluorescence intensity. The presence of a tag is detected by hybridization of a probe to the tag producing above threshold fluorescence intensity measured at the wavelength of the probe corresponding to the tag. The table at the right in FIG. 1 shows different combinations of above threshold fluorescence intensities (shown as different shaded circles and marked in the right column as “1”) for the exemplary four colors corresponding to the exemplary four tags used in the exemplary design. Below threshold fluorescence intensities (shown as transparent circles and marked in the right column as “0”) represent the absence of a tag in a given tag region. Each group of four digits in the right column represents the digital signature of a barcode. There are eleven possible different digital signatures corresponding to the eleven different barcodes available for a barcode set including four tag regions.

A labeling region including the plurality of tags or tag regions is of sufficient length to include the tags, and be readily and confidently amplifiable between a single pair of primers. A labeling region is optionally from 50 to 2000 nucleotides in length or any value or range there between. A labeling region is optionally 50 to 1000, 100 to 1000, 100 to 750, 50 to 500, or 50 to 750 nucleotides long. Excellent results are regularly achieved using a labeling region of 100 to 300 nucleotides in length. Using currently available polymerases, a labeling region has a maximum length of 2500 nucleotides, but this is envisioned to possibly be lengthened by use of forthcoming materials or processes for amplification between a single pair of primers.

A labeling region is amplified by a single pair of primers that each hybridize to a region flanking the tags within a labeling region. The design of primers is routinely achieved by one of ordinary skill in the art. Primer design is optionally achieved by inputting a known nucleotide sequence into a primer design tool to obtain possible primers. One such primer design tool is the NCBI/Primer-BLAST available from National Institutes of Health and freely accessible using the internet. An additional common tool for primer design is the Primer3 freely available on the internet from the Massachusetts Institute of Technology.

The introduction of mutations in a labeling region to produce a tag including a synonymous mutation is achieved by markerless recombination methods. One such method is described by Tischer B K, et al., Biotechniques, 2006; 40(2):191-197. Other techniques are known in the art.

Numerous methods are known in the art for the synthesis and production of nucleic acid sequences that can be used to introduce a tag to an insertion site or labeling region in an organism's genome. A tag, plurality of tags, or an entire labeling region or insertion region may be synthetically synthesized by techniques known in the art, illustratively, solid phase synthesis using a phosphoramidite method. The synthesized tag sequence may then be inserted (optionally following amplification) into the genome of a host organism by markerless recombination methods to introduce the tag sequence into the labeling region, or optionally by other insertion methods known in the art.

A tag, plurality of tags, or labeling region may be amplified prior to insertion. Amplification may be by any suitable technique illustratively including cloning and expression in cells such as E. coli, insect cells such as Sf9 cells, yeast, and mammalian cell types such as Hela cells, Chinese hamster ovary cells, or other cells systems known in the art as amendable to transfection and nucleic acid and/or protein expression. Methods of nucleic acid isolation are similarly recognized in the art. Illustratively, plasmid DNA amplified in E. coli is cleaved by suitable restriction enzymes such as NdeI and XhoI to linearize DNA. The DNA is subsequently isolated following gel electrophoresis using a S.N.A.P.™ UV-Free Gel Purification Kit (Invitrogen, Carlsbad, Calif.) as per the manufacturer's instructions.

Numerous agents are amenable to facilitate cell transfection illustratively including synthetic or natural transfection agents such as LIPOFECTIN, baculovirus, naked plasmid or other DNA, or other systems known in the art.

The introduced synonymous substitutions should not be recognized by the restriction modification system of the host organism. For example, a substitution in a stealth barcode tag or insertion sequence tag should not introduce a new restriction site recognized by a restriction enzyme produced by the host organism. Alternatively or in addition, regions near the tag or the tag itself may incorporate a methyl group so as to prevent binding by a host restriction enzyme thereby preventing cleavage of the tag or the labeling region. Target sites for restriction enzymes are known in the art and determining whether a mutation will introduce a recognized restriction site is readily achieved by comparison of the mutated sequence with the target sequences of known restriction enzymes.

A labeling region is in a gene of an organism in the case of a stealth barcode, or in a non-coding region of the genome of an organism in the case of an insertion barcode. In the case of a stealth barcode, a tag is present in an essential, coessential, conditionally essential, or phenotype supporting gene. Essential genes are those that are indispensible for the support of the life or life cycle of the organism. Essential genes are understood for many organisms, and are expected to be similar for other organisms. A database of essential genes is available in the Database of Essential Genes taught in Ren Zhang and Yan Lin, Nucleic Acids Research 2009; 37: D455-D458; and Zhang et al., Nucleic Acids Research, 2004; 32: D271-D272. Methods of identifying coessential, conditionally essential, or phenotype supporting genes are also known in the art.

A process optionally includes amplifying the entire plurality of tags by hybridizing a forward primer to the labeling region or area adjacent thereto, and hybridizing a reverse primer to a second site in the labeling region or area adjacent thereto, and subjecting the labeling region to PCR amplification producing an amplification product including all of the tags or tag regions. One process of in vitro amplification is the polymerase chain reaction (PCR) such as that described in U.S. Pat. Nos. 4,683,202 and 4,683,195. The term “polymerase chain reaction” refers to a process for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. Many PCR processes are known to those of skill in the art and may be used in the process of the invention. For example, DNA is subjected to 30 to 35 cycles of amplification in a thermocycler as follows: 95° C. for 30 see, 52 to 60° C. for 1 min, and 72° C. for 1 min, with a final extension step of 72° C. for 5 min. For another example, DNA is subjected to 35 polymerase chain reaction cycles in a thermocycler at a denaturing temperature of 95° C. for 30 sec, followed by varying annealing temperatures ranging from 54 to 58° C. for 1 min, an extension step at 70° C. for 1 min, with a final extension step at 70° C. for 5 min. The parameters of PCR cycling times, temperature, and number of steps are dependent on the primer pair, their melting temperature, and other considerations understood by those of ordinary skill in the art. It is appreciated that optimizing PCR parameters for various probe sets is well within the skill of the art and is performed as mere routine optimization.

Amplification of a labeling region and the tags contained therein yields an amplification product. PCR amplification is preferred for detection of the barcode (e.g. tags) encoded by the labeling region in a target subject. The term “amplifying” or “amplified” defines the process of making multiple copies of the nucleic acid from a single or lower copy number of nucleic acid sequence molecule. The amplification of nucleic acid sequences is carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, AmpliTaq Gold DNA Polymerase from Applied Biosystems, other available DNA polymerases, reverse transcriptase (preferably iScript RNase H+ reverse transcriptase), ligase, and other enzymes, including heat-stable enzymes (i.e., those enzymes that perform primer extension after being subjected to temperatures sufficiently elevated to cause denaturation). In a preferred embodiment, the enzyme is hot-start iTaq DNA polymerase from Bio-rad (Hercules, Calif.). Suitable enzymes will facilitate combination of the nucleotides in the proper manner to form the primer extension products that are complementary to each mutant nucleotide strand. Generally, the synthesis is initiated at the 3′-end of each primer and proceed in the 5′-direction along the template strand, until synthesis terminates, producing molecules of different lengths. There may be amplification agents, however, that initiate synthesis at the 5′-end and proceed in the other direction, using the same process as described above. In any event, the process of the invention is not to be limited to the embodiments of amplification described herein.

Primers used according to the process of the invention are complementary to each strand of nucleotide sequence to be amplified. The term “complementary” means that the primers must hybridize with their respective strands under conditions that allow the agent for polymerization to function. In other words, the primers that are complementary to the flanking sequences hybridize with the flanking sequences and permit amplification of the nucleotide sequence. Preferably, the 3′ terminus of the primer that is extended is perfectly base paired with the complementary flanking strand.

Those of ordinary skill in the art will know of various amplification processes that can also be utilized to increase the copy number of target nucleic acid sequence. The nucleic acid sequences detected in the process of the invention are optionally further evaluated, detected, cloned, sequenced, and the like, either in solution or after binding to a solid support, by any process usually applied to the detection of a specific nucleic acid sequence such as another polymerase chain reaction, oligomer restriction (Saiki et al., BioTechnology 3:1008 1012 (1985)), allele-specific oligonucleotide (ASO) probe analysis (Conner et al., PNAS 80: 278 (1983)), oligonucleotide ligation assays (OLAs) (Landegren et al., Science 241:1077 (1988)), RNase Protection Assay and the like.

Detection of the presence or absence of a tag for the generation of a barcode readout are achieved using a plurality of probes each specific for a single tag sequence. A probe is illustratively a labeled oligonucleotide, an antibody, or other suitable composition capable of specifically recognizing a nucleic acid sequence such as an aptamer. Probe size or length is dictated by the size or length of the tag that it will be specific for. Optionally, a probe is an oligonucleotide with a length identical to that of its target tag sequence. Optionally, a probe has a length that is longer, equal to, or shorter than a tag, with the proviso that the probe be capable of selectively hybridizing or otherwise recognizing the target tag to the exclusion of other tags in the labeling region or to non-tag sequences. In preferred embodiments, a probe has a length that is longer or equal to a tag to which it hybridizes.

In some embodiments, a fluorescent reporter dye, such as FAM dye (illustratively 6-carboxyfluorescein), is covalently linked to the 5′ end of the oligonucleotide probe or other location on an alternate probe type. Other dyes illustratively include TAMRA, AlexaFluor dyes such as AlexaFluor 495 or 590, Cascade Blue, Marina Blue, Pacific Blue, Oregon Green, Rhodamine, Fluorescein, TET, HEX, Cy5, Cy3, Quasar670, and Tetramethylrhodamine. Each of the reporters is quenched by a quencher at the 3′ end or other non-fluorescent quencher. Quenching molecules are suitably matched to the fluorescence maximum of the dye. Any suitable fluorescent probe for use in real-time PCR detection systems is illustratively operable in the instant invention. Similarly, any quenching molecule for use in real-time PCR systems is illustratively operable. In a preferred embodiment, a 6-carboxyfluorescein reporter dye is present at the 5′-end and matched to BLACK HOLE QUENCHER (BHQ1, Biosearch Technologies, Inc., Novato, Calif.). The fluorescence signals from these reactions are captured at the end of extension steps as PCR product is generated over a range of the thermal cycles, thereby allowing the quantitative determination of the bacterial load in the sample based on an amplification plot.

In some embodiments, the processes of amplification and detection are achieved simultaneously such as in a real-time PCR assay or quantitative real-time PCR using labeled oligonucleotide probes. In a preferred embodiment, the quantitative PCR used in the present invention is a TaqMan assay (Holland et al., PNAS 88(16):7276 (1991)). It is appreciated that the current invention is amenable to performance on other real-time PCR systems and in protocols that use alternative reagents illustratively including, but not limited to, Molecular Beacons probes, Scorpion probes, multiple reporters for multiplex PCR, combinations thereof, or other DNA or RNA detection methods.

Quantitative real time PCR (qPCR) is characterized by sensitivity (the ability to detect the matching template), specificity (the ability to reject the mismatching template) and selectivity (the ability to detect small number of copies of the matching template when large number of copies of the mismatching template is present). The performance of qPCR assays is dependent on all three characteristics. Digital PCR as used in the present invention eliminates the dependence of qPCR performance on selectivity. In some embodiments of the present invention, digital PCR is used as threshold detection method where assay performance depends on sensitivity for the detection of a tag and specificity for the rejection of mismatching templates all while keeping the threshold for tag detection at low levels.

The assays are optionally performed on an instrument designed to perform such assays, for example those available from Applied Biosystems (Foster City, Calif.). In more preferred specific embodiments, the present invention provides a real-time quantitative PCR assay to detect the presence of one or more tags present or absent in a target nucleic acid sequence in a sample by subjecting the nucleic acid from the sample to PCR reactions using specific primers, and detecting the amplified product using a plurality of probes each specifically directed to a single tag. In preferred embodiments, the probe is a TaqMan probe which consists of an oligonucleotide with a 5′-reporter dye and a 3′-quencher dye. It is appreciated that a probe may have other configurations as are well understood in the art.

A variety of methods are available for barcode detection including fluorescence, mass spectrometry, microarrays, sequencing, PCR etc. New generation of high throughput compatible digital methods for nucleic acids analysis have emerged, which has significantly expanded the utility of genomic methods. However, in the field of cellular barcoding these systems have been underutilized because the commonly used barcode design does not recognize the digital capabilities of the detecting instruments.

At the present time there are five commercially available platforms for digital PCR. These include: a) two platforms offered by Fluidigm corporation where one of the platforms is real time capable and both platforms have four colors; b) one platform offered by Life Technologies with real time capabilities and two colors, c) one platform offered by Bio-Rad Laboratories with end point detection and two colors and d) one platform offered by RainDance with end point detection and two colors. The design of the digital PCR reactions allows for single color multiplexing. Two-plex per color in a five-plex assay has been demonstrated using the two color digital PCR platform from RainDance. Therefore, theoretically eight-plex reactions are possible on a four color instrument. The present art allows the performance of up to five-plex digital PCR reactions. The four-color digital PCR instruments from Fluidigm are the most advanced digital PCR platform available. The examples shown in FIG. 1 are for a four color single-plex per color digital PCR instruments such as the ones made by Fluidigm Co. The Roche LightCycler 480 real time PCR instrument has optical filter combinations, which allow for the simultaneous detection of five fluorescent channels. This shows that the construction of five color digital PCR instruments is possible. The rules for barcode set design of the invention allow for designing barcode sets for instrument with any number of channels. For example, a barcode set designed for five channel digital PCR detection allows for simultaneous detection of up to twenty six different barcoded strains.

Returning to FIG. 1, the barcode region is amplified using a single pair of primers (regions shaded at ends). There are four common tag positions chosen for this barcode set. A tag might be placed on one of the tag positions or the tag position can be left without tag. The presence of a tag at a given tag position is detected as above threshold fluorescence intensity from a tag-specific hybridization probe (e.g. TaqMan or other probe). The absence of a tag at the same position is detected as below threshold fluorescence intensity from the corresponding tag-binding probe. Each probe is distinguished by a dedicated color (e.g. fluorescent label). In the design used in FIG. 1, four probes with four different colors are used. At least two tags are used per barcode. The presence of a tag is detected by measuring any of the combinations of above threshold fluorescence intensities illustratively as is shown in the table at the right in FIG. 1. The shaded circles represent above threshold fluorescence signals generated by the probes bound to the tags (e.g. fluorescence after TaqMan hydrolysis). The transparent circles (shown as white-filled circles) represent below threshold fluorescence intensity measured in the absence of a tag. The right column of the table shows the digital signature corresponding to each barcode.

Theoretically, the number of simultaneously detectable tags (i.e. the size of the overall barcode) is limited only by the instrumentation used to detect the signals. As such, it is appreciated that the invention is not limited by the number of tags, but may, in some embodiments use 3, 4, 5, 6, 7, 8, or more tags in a single barcode. Table 1 illustrates Digital signatures for barcode sets for use with three, four and five channel digital PCR instruments. Four different barcoded strains can be simultaneously detected in a mixed population using three channel digital PCR instruments. The number of simultaneously detected barcoded strains increases to eleven for a four channel digital PCR and to twenty six for a five color digital PCR instruments, correspondingly.

TABLE 1
Exemplary digital barcode signatures

| Digital signatures for | Digital signatures for | | Digital signatures for | | | |
|---|---|---|---|---|---|---|
| barcode sets using | barcode sets using | | barcode sets using | | | |
| three tags | four tags | | five tags | | | |
| 110 | 1100 | 1110 | 11000 | 00110 | 10101 | 11101 |
| 101 | 1010 | 1101 | 10100 | 00101 | 10011 | 11011 |
| 011 | 1001 | 1011 | 10010 | 00011 | 01110 | 10111 |
| 111 | 0110 | 0111 | 10001 | 11100 | 01101 | 01111 |
| | 0101 | 1111 | 01100 | 11010 | 01011 | 11111 |
| | 0011 | | 01010 | 11001 | 00111 | |
| | | | 01001 | 10110 | 11110 | |

Also provided are insertion barcodes and processes of detecting or labeling organisms with one or more insertion barcodes by the insertion of a non-native oligonucleotide into a portion of the non-coding region of the organism's genome. Similar to stealth barcodes, the presence of an insertion barcode does not alter the phenotype of the receiving organism.

Processes using insertion barcodes include: selecting an insertion site in a non-coding region of a genome of an organism; inserting at the insertion site a plurality of nucleotides within said non-coding region by markerless recombination to produce an insertion sequence including a plurality of tags, the plurality of tags constituting a barcode or portion thereof; and optionally amplifying the insertion sequence by a polymerase chain reaction using a single pair of primers including a forward primer and a reverse primer to produce an amplification product. Selecting an insertion site is optionally achieved by the process of Buckley, P., et al, Applied and Environmental Microbiology, 2012; 78:8272-8280. Unique tag sequences are selected and an oligonucleotide including the tag sequences and primer hybridization sites for subsequent amplification is produced and then inserted at the insertion site by markerless recombination or other method that does not result in recognition of the inserted sequence by the organism's restriction modification system and does not alter the organism's fitness in the conditions of intended use.

Following creation of one or more stealth barcodes (i.e. tags), or insertion barcodes, a genomic analysis is optionally performed to confirm that the genomic manipulations during the barcode insertion have not resulted in the loss or gain of undesirable genetic material. Such analyses optionally include DNA sequencing such as Sanger sequencing. Alternatively, for a stealth barcode, a protein encoded by the tagged gene is isolated and sequenced or otherwise analysed for wild-type protein sequence. The combination of DNA sequencing with protein sequencing will confirm both proper incorporation of the tag sequences and the phenotype neutrality for a stealth barcode. Optionally, protein levels are similarly analyzed. Optionally, an organism is subjected to phenotypic challenge to determine if it behaves as a non-labeled organism in the conditions of the desired usage.

Overall, several criteria are used for barcodes of the stealth type and/or the insertion type. A barcode must include at least two tags. It is appreciated that a barcode may be formed with a single tag, but a single tag is typically insufficient for digital PCR analyses and lacks power to differentially label several organisms and perform a multiplex reaction to simultaneously detect the presence or absence of all tagged organisms.

Each tag is recognized by a dedicated probe with specific label (or intensity range when two-plex detection per color is used). All probes corresponding to a given barcode set are used together in a multiplex format during barcode detection using digital PCR analyses.

The amplification of the nucleic acid sequences containing all tag loci assigned to a given barcode set is under the control of a single pair of primers. This radically simplifies the system, yet is optional.

All barcodes are phenotype neutral. The phenotype-neutrality of the “insertion barcodes” is ensured by their insertion in the intergenic space using the following rules: 1) the target region for barcode insertion must be located in the chromosome; 2) the insertion point must lie near the midpoint of an intergenic space larger than 500 bp; 3) no annotated genes or potential ORFs in the intergenic space should be present at or close to the target region; 4) the target region must lie between two convergently transcribed genes; 5) no repetitive structure in intergenic space should be present at the insertion site; 6) no identical repetitive elements >200 bp in size should be present within 10000 bp of the target region; 7) target must be intact and consistently annotated in two or more available sequences of closely related strains. Other rules of insertion providing phenotype neutrality can also be used for the insertion of the “insertion barcodes.”

Stealth barcodes maintain phenotype neutrality yet are encoded in essential, coessential genes, conditionally essential genes, or phenotype supporting genes. This ensures barcode maintenance because mutations of essential or coessential genes are usually lethal and the maintenance of phenotype supporting genes is enforced by selective growth conditions.

Finally, the unique sequences used in the design of “insertion barcodes” or the synonymous substitutions used in the design of “stealth barcodes” should not be recognized by the restriction-modification system of the barcoded host organism. Following the above parameters will ensure phenotype neutral barcoding of a subject and promote maintenance of the barcode in the population.

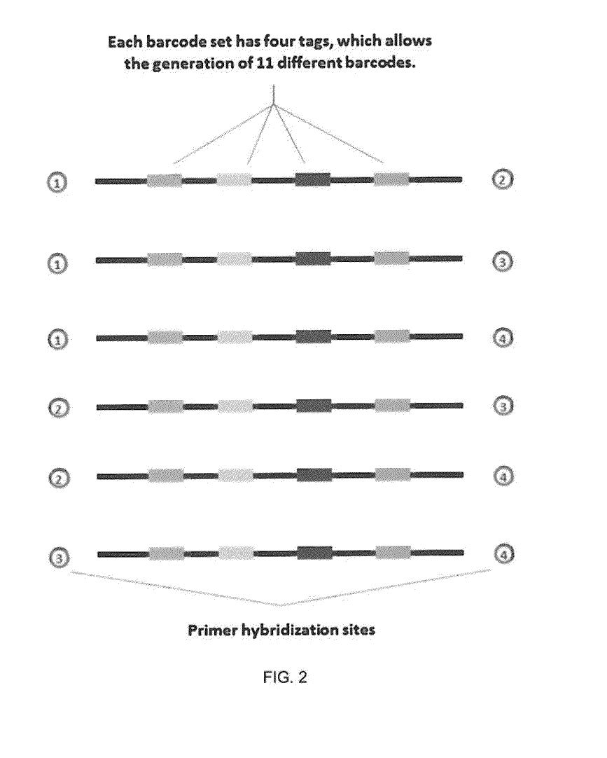

It is another aspect of the invention that a modular design may be employed in barcode design. The ability to generate a large number of phenotype neutral barcoded strains is limited by the ability to identify suitable sites for introduction of barcode sets. One of the challenges is the identification of loci for tag insertion and identifying tag sequences, which are compatible with the host genomic machine. Therefore, it is desirable to develop strategy for the design of a large number of barcodes using small number of host-compatible elements. The combinatorial approach described in the previous sections allows for significant increase of the numbers of barcodes per set using small number of tags; however the overall number of barcodes is still comparable with the number of available channels of the digital PCR instrument used for detection. The extension of the combinatorial approach for barcode design to include the unique sequences for primer hybridization allows for further increase of the number of barcodes designed from a small number of host-compatible unique sequences. The design extending the combinatorial approach to include the primer hybridization sequences is called modular design. An example of modular barcode design using eight host-compatible unique sequences is shown in FIG. 2.

As shown in FIG. 2, eight unique sequences are identified as modular sequences, where four sequences are used as tags (marked with four different colors) and four are used as primer hybridization sites (marked with numbers). The tag modules of each set are used in different combinations as illustrated in FIG. 1. Using different combinations of primer hybridization sites, up to six barcode sets are available for the selected eight modules, which corresponds to 66 different barcodes.

At least two phenotype neutral insertion sites are available in the B. thuringiensis chromosome. Other or similar phenotype neutral insertion sites are available in the chromosomes of other organisms of interest. At least six unique sequences are necessary for designing a four-tag barcode set; two for primer hybridization sites and four for tags. More than 4,000 sequences have been identified as potential unique sequences in the universal TagModule collection (Oh, J., et al, Nucleic Acids Research, 2010; 38:e146.). For the purpose of this example eight unique sequences are chosen as modules for four-channel modular barcode set design. Four of the modules are used as tags and four are used as primer hybridization sites (FIG. 2). Theoretically, up to 66 barcoded strains are possible using eight modules (6 sets with 11 barcoded strains per set as shown in FIG. 2). For this design, simultaneous barcode detection is limited to one barcode set. The modular barcode design has some unique advantages. In addition to the ability to provide large number of barcoded strains as already discussed it allows to design barcode sets over a range of organisms.

As such, in some embodiments, a plurality of barcode sets is introduced either as insertion barcodes, or as stealth barcodes, or combinations thereof in one or a plurality of organisms. Each barcode is under the control of a single primer pair and the combination of two or more primer pairs allows for the detection of multiple barcodes simultaneously or sequentially.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, NY, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, NY, 1991; and Methods of Immunological Analysis, ed. Masseyeff et al., John Wiley & Sons, NY, 1992.

Various aspects of the present invention are illustrated by the following non-limiting examples. The examples are for illustrative purposes and are not a limitation on any practice of the present invention. It will be understood that variations and modifications can be made without departing from the spirit and scope of the invention. A person of ordinary skill in the art readily understands where any and all necessary reagents may be commercially obtained.

### EXAMPLES

### Example 1

Design of a four-tag “stealth barcode” set for B. thuringiensis BMB171 and related species using the spo0A gene.

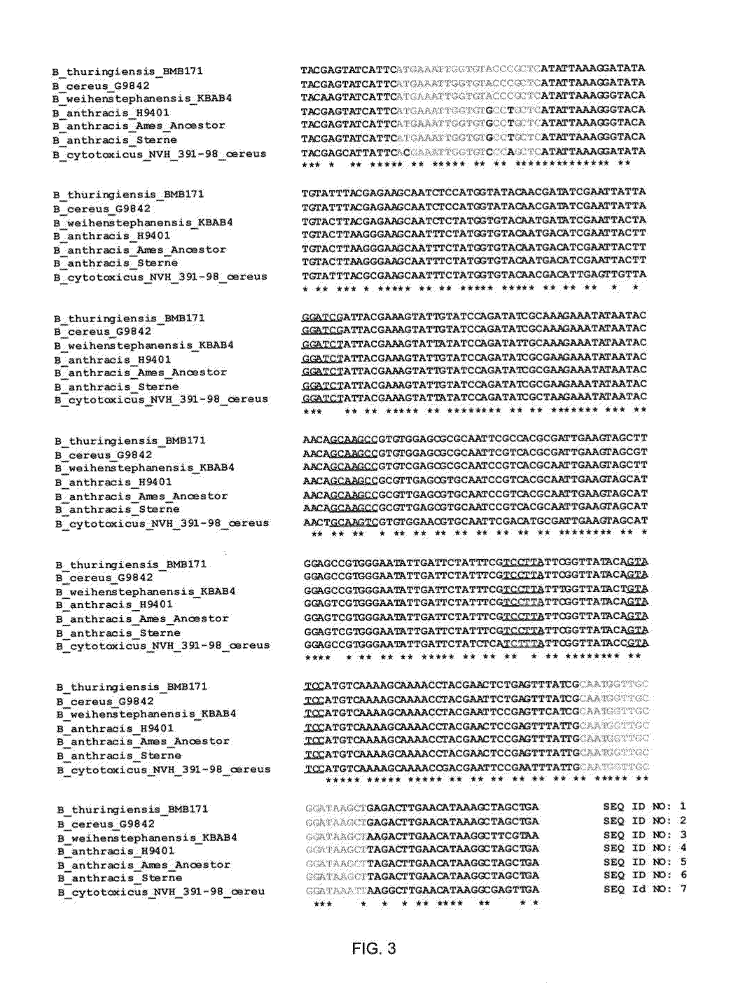

The Spo0A_C conserved domain of Spo0A is chosen for this exemplary barcode design. Partial nucleic acid sequences close to the stop codon are aligned using ClustalW for several closely related species (FIG. 3). The use of conserved domains allows the design of barcode sets crossing over several species. This is illustrated in the example shown in FIG. 2, where the primer pair controlling the amplification of the region containing the four tags of the set is common for seven closely related species and subspecies (with some degree of degeneracy of the forward primer), including: B. thuringiensis, B. cereus, B. anthracis (represented with three subspecies), B. weihenstephanensis, and B. cytotoxicus.

The following four tags are used in this example: a) GGCTCC-tag substituting for GGATCG; b) GCCTCG-tag substituting for GCAAGC; c) AGCCTT-tag substituting for TCCTTA; and d) GTTTCT-tag substituting for GTATCC. For stealth barcode sets crossing over multiple species, multiple probes per tag region can be used provided that each barcode has unique digital signature and all barcodes from the set are amplified using the same primer pair (some level of primer degeneracy is allowed in the design as shown in FIG. 1).

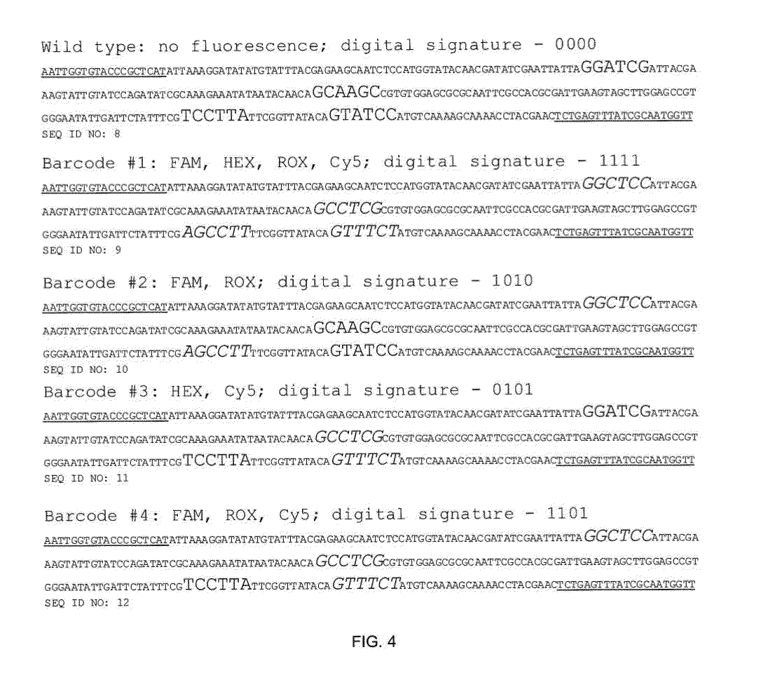

An exemplary barcode set used for B. thuringiensis is depicted in FIG. 4. The underlined sequences represent the primer hybridization sites. The tags are marked with in large capital letter, each with one of the commonly used fluorescence probes in four color digital PCR instruments as indicated. The digital signature corresponding to each barcode is shown at the right of the header to each barcode. The chosen color scheme is ordered as follows: FAM, HEX, ROX, Cy5. The digital signature of the wild type strain (top raw) is (0000). The digital signatures of the four barcodes shown in this example are: #1 (1111), #2 (1010), #3 (0101), and #4 (1101). There are seven additional barcodes from this set, which are not shown, but are readily envisioned such as depicted in Table 1.

It is appreciated that all reagents are obtainable by sources known in the art unless otherwise specified.

### REFERENCES

1. Baker, M. 2012. Digital PCR hits its stride. Nat Meth 9:541-544.
2. Buckley, P., B. Rivers, S. Katoski, M. H. Kim, F. J. Kragl, S. Broomall, M. Krepps, E. W. Skowronski, C. N. Rosenzweig, S. Paikoff, P. Emanuel, and H. S. Gibbons. 2012. Genetic Barcodes for Improved Environmental Tracking of an Anthrax Simulant. Applied and Environmental Microbiology 78:8272-8280.
3. Hensel, M., J. Shea, C. Gleeson, M. Jones, E. Dalton, and D. Holden. 1995. Simultaneous identification of bacterial virulence genes by negative selection. Science 269:400-403.
4. Krutzik, P. O., and G. Nolan. 2011. Multiplex cellular assays using detectable cell barcodes. U.S. Pat. No. 8,003,312.
5. Oh, J., E. Fung, M. N. Price, P. S. Dehal, R. W. Davis, G. Giaever, C. Nislow, A. P. Arkin, and A. Deutschbauer. 2010. A universal TagModule collection for parallel genetic analysis of microorganisms. Nucleic Acids Research 38:e146.
6. Roth, F. P., Y. Suzuki, and J. Mellor. 2012. Methods and applications for stitched DNA barcodes U.S. Pat. No. 8,268,564.
7. Schwartz, D. C., K. D. Potamousis, S. Zhou, S. J. Goldstein, M. A. Newton, R. A. Runaheim, D. K. Forrest, and C. P. Churas. 2012. Methods of whole genome analysis. U.S. Pat. No. 8,221,973.
8. Shoemaker, D. D., D. A. Lashkari, D. Morris, M. Mittmann, and R. W. Davis. 1996. Quantitative phenotypic analysis of yeast deletion mutants using a highly parallel molecular bar-coding strategy. Nat Genet 14:450-456.
9. Sydney, B. 2012. Methods and compositions for tagging and identifying polynucleotides U.S. Pat. No. 8,168,385.
10. Winzeler, E. A., D. D. Shoemaker, A. Astromoff, H. Liang, K. Anderson, B. Andre, R. Bangham, R. Benito, J. D. Boeke, H. Bussey, A. M. Chu, C. Connelly, K. Davis, F. Dietrich, S. W. Dow, M. El Bakkoury, F. Foury, S. H. Friend, E. Gentalen, G. Giaever, J. H. Hegemann, T. Jones, M. Laub, H. Liao, N. Liebundguth, D. J. Lockhart, A. Lucan-Danila, M. Lussier, N. M'Rabet, P. Menard, M. Mittmann, C. Pai, C. Rebischung, J. L. Revuelta, L. Riles, C. J. Roberts, P. Ross-MacDonald, B. Scherens, M. Snyder, S. Sookhai-Mahadeo, R. K. Storms, S. Véroaneau, M. Voet, G. Volckaert, T. R. Ward, R. Wysocki, G. S. Yen, K. Yu, K. Zimmermann, P. Philippsen, M. Johnston, and R. W. Davis. 1999. Functional Characterization of the S. cerevisiae Genome by Gene Deletion and Parallel Analysis. Science 285:901-906.
11. Zhelev, D. V., M. Hunt, A. Le, C. Dupuis, S. Ren, and H. S. Gibbons. 2012. Effect of the Bacillus atrophaeus subsp. globigii Spo0F H101R Mutation on Strain Fitness. Applied and Environmental Microbiology 78:8601-8610.
12. Zhong, Q., S. Bhattacharya, S. Kotsopoulos, J. Olson, V. Taly, A. D. Griffiths, D. R. Link, and J. W. Larson. 2011. Multiplex digital PCR: breaking the one target per color barrier of quantitative PCR. Lab on a Chip 11:2167-2174.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process of labeling an organism comprising:
selecting a labeling region within an exon of a gene;
substituting a plurality of non-wild type nucleotides within said labeling region to produce a plurality of tags within said labeling region, said plurality of tags representing a barcode, at least two of said plurality of tags each in a separate codon resulting in synonymous substitutions at each nucleotide substitution site; said step of substituting producing silent mutations in said gene such that a protein encoded by said gene is the wild-type amino acid sequence.

2. The process of claim 1 wherein said labeling region includes two primer binding sites flanking said plurality of tags; said primer binding sites suitable for amplification of said labeling region by a single pair of primers to produce an amplification product.

3. The process of claim 1 wherein at least four non-wild type nucleotides are substituted in said plurality of tags.

4. The process of claim 1 further comprising amplifying the entire plurality of tags by hybridizing a forward primer to said labeling region, and hybridizing a reverse primer to said labeling region; and
subjecting said labeling region to PCR amplification producing an amplification product comprising all of said tags.

5. The process of claim 4 further comprising, contacting a plurality of probes to said amplification product; and
detecting the presence or absence of hybridization of each of said probes to said tags.

6. The process of claim 1 wherein said gene is an essential, coessential, conditionally essential, or phenotype supporting gene, the presence of said tags not altering the phenotype of said organism.

7. A process of identifying an organism in a sample comprising:
producing an amplification product by amplifying a nucleotide sequence comprising two or more tags, at least two of said tags being non-wild type in nucleotide sequence, using a forward primer that hybridizes to a first region within a labeling region within a genome of said organism, and a reverse primer that hybridizes to a second region within said labeling region, under conditions suitable for a polymerase chain reaction;
contacting said amplification product with a plurality of probes each specific to one of said tags;
detecting the presence or absence of each of said tags in said amplification product by hybridization of one or more of said probes to said amplification product to produce a barcode readout; and
identifying said organism by the constitution of said barcode readout.

8. The process of claim 7 wherein said step of contacting is by contacting four or more probes.

10. The process of claim 7 wherein said step of contacting is by contacting five or more probes.

11. The process of claim 7 wherein each of said probes comprises a unique label.

12. The process of claim 7 wherein said nucleotide sequence is entirely within a gene exon of said organism.

13. The process of claim 12 wherein said gene is essential, coessential, conditionally essential, or phenotype supporting.

14. The process of claim 7 wherein each of said tags comprises a synonymous nucleotide substitution.

15. A process of labeling an organism comprising:
selecting an insertion site in a non-coding region of a genome of an organism;
inserting at said insertion site a plurality of nucleotides within said non-coding region to produce an insertion sequence comprising a plurality of tags, said plurality of tags constituting a barcode or portion thereof;
said insertion sequence not recognized by a restriction modification system;
said insertion sequence producing phenotype neutrality in said organism;
amplifying said insertion sequence by a polymerase chain reaction using a single pair of primers comprising a forward primer and a reverse primer to produce an amplification product;
contacting a plurality of probes with said amplification product; and
detecting the presence or absence of hybridization of each of said probes to a corresponding tag by digital analyses.

16. The process of claim 15 wherein said non-coding region is 500 nucleotides or larger.

17. The process of claim 15 wherein said non-coding region is flanked by convergently transcribed genes.

18. The process of claim 15 wherein said non-coding region is free from identical repetitive elements of 200 nucleotides or more within 10,000 nucleotides from said insertion site.

19. The process of claim 15 further comprising generating a barcode from detecting the presence or absence of said tags, said barcode readable as a digital output

20. The process of claim 15 wherein said step of inserting is by markerless recombination.


SEQUENCE LISTING (12 sequences)

SEQ ID NO: 1 - DNA, 334 bp, B_thuringiensis_BMB171
tacgagtatcattcatgaaattggtgtacccgctcatattaaaggatatatgtatttacgagaagcaatctccatggtatacaacgatatcgaattattaggatcgattacgaaagtattgtatccagatatcgcaaagaaatataatacaacagcaagccgtgtggagcgcgcaattcgccacgcgattgaagtagcttggagccgtgggaatattgattctatttcgtccttattcggttatacagtatccatgtcaaaagcaaaacctacgaactctgagtttatcgcaatggttgcggataagctgagacttgaacataaagctagctga

SEQ ID NO: 2 - DNA, 334 bp, B_cereus_G9842
tacgagtatcattcatgaaattggtgtacccgctcatattaaaggatatatgtatttacgagaagcaatctccatggtatacaacgatatcgaattattaggatcgattacgaaagtattgtatccagatatcgcaaagaaatataatacaacagcaagccgtgtggagcgcgcaattcgtcacgcgattgaagtagcgtggagccgtgggaatattgattctatttcgtccttattcggttatacagtatccatgtcaaaagcaaaacctacgaattctgagtttatcgcaatggttgcggataagctgagacttgaacataaagctagctga

SEQ ID NO: 3 - DNA, 334 bp, B_weihenstephanensis_KBAB4
tacaagtatcattcatgaaattggtgtacccgctcatattaaagggtacatgtacttacgagaagcaatctctatggtgtacaatgatatcgaattactaggatctattacgaaagtattatatccagatattgcaaagaaatataatacaacagcaagccgtgtcgagcgcgcaatccgtcacgcaattgaagtagcttggagccgtgggaatattgattctatttcgtccttatttggttatactgtatccatgtcaaaagcaaaacctacgaattccgagttcatcgcaatggttgcggataagctaagacttgaacataaggcttcgtaa

SEQ ID NO: 4 - DNA, 334 bp, B_anthracis_H9401
tacgagtatcattcatgaaattggtgtgcctgctcatattaaagggtacatgtacttaagggaagcaatttctatggtgtacaatgacatcgaattacttggatctattacgaaagtattgtatccagatatcgcgaagaaatataatacaacagcaagccgcgttgagcgtgcaatccgtcacgcaattgaagtagcatggagtcgtgggaatattgattctatttcgtccttattcggttatacagtatccatgtcaaaagcaaaacctacgaactccgagtttattgcaatggttgcggataagcttagacttgaacataaggctagctga

SEQ ID NO: 5 - DNA, 334 bp, B_anthracis_Ames_Ancestor; identical to SEQ ID NO: 4

SEQ ID NO: 6 - DNA, 334 bp, B_anthracis_Sterne; identical to SEQ ID NO: 4

SEQ ID NO: 7 - DNA, 334 bp, B_cytotoxicus_NVH_391-98_cereus
tacgagcattattcacgaaattggtgtcccagctcatattaaaggatatatgtatttacgcgaagcaatttctatggtgtacaacgacattgagttgttaggatctattacgaaagtattatatccagatatcgctaagaaatataatacaactgcaagtcgtgtggaacgtgcaattcgacatgcgattgaagtagcatggagccgtgggaatattgattctatctcatctttattcggttataccgtatccatgtcaaaagcaaaaccgacgaattccgaatttattgcaatggttgcggataaattaaggcttgaacataaggcgagttga

SEQ ID NO: 8 - DNA, 280 bp, B_thuringiensis_BMB171
aattggtgtacccgctcatattaaaggatatatgtatttacgagaagcaatctccatggtatacaacgatatcgaattattaggatcgattacgaaagtattgtatccagatatcgcaaagaaatataatacaacagcaagccgtgtggagcgcgcaattcgccacgcgattgaagtagcttggagccgtgggaatattgattctatttcgtccttattcggttatacagtatccatgtcaaaagcaaaacctacgaactctgagtttatcgcaatggtt

SEQ ID NO: 9 - DNA, 280 bp, Artificial Sequence
  Sequence from B_thuringiensis_BMB171 with four synonymous substitutions, where codon pairs GGCTCC (83 to 88), GCCTCG (138 to 143), AGCCTT (212 to 217) and GTTTCT (230 to 235) replace wild type codon pairs GGATCG, GCAAGC, TCCTTA and GTATC
aattggtgtacccgctcatattaaaggatatatgtatttacgagaagcaatctccatggtatacaacgatatcgaattattaggctccattacgaaagtattgtatccagatatcgcaaagaaatataatacaacagcctcgcgtgtggagcgcgcaattcgccacgcgattgaagtagcttggagccgtgggaatattgattctatttcgagccttttcggttatacagtttctatgtcaaaagcaaaacctacgaactctgagtttatcgcaatggtt

SEQ ID NO: 10 - DNA, 280 bp, Artificial Sequence
  Sequence from B_thuringiensis_BMB171 with two synonymous substitutions, where codon pairs GGCTCC (83 to 88) and AGCCTT (212 to 217) replace wild type codon pairs GGATCG and TCCTTA.
aattggtgtacccgctcatattaaaggatatatgtatttacgagaagcaatctccatggtatacaacgatatcgaattattaggctccattacgaaagtattgtatccagatatcgcaaagaaatataatacaacagcaagccgtgtggagcgcgcaattcgccacgcgattgaagtagcttggagccgtgggaatattgattctatttcgagccttttcggttatacagtatccatgtcaaaagcaaaacctacgaactctgagtttatcgcaatggtt

SEQ ID NO: 11 - DNA, 280 bp, Artificial Sequence
  Sequence derived from the B_thuringiensis_BMB171 with introduction of two synonymous substitutions.  Codon pairs GCCTCG (138 to 143) and GTTTCT (230 to 235) replace wild type codon pairs GCAAGC and GTATCC.
aattggtgtacccgctcatattaaaggatatatgtatttacgagaagcaatctccatggtatacaacgatatcgaattattaggatcgattacgaaagtattgtatccagatatcgcaaagaaatataatacaacagcctcgcgtgtggagcgcgcaattcgccacgcgattgaagtagcttggagccgtgggaatattgattctatttcgtccttattcggttatacagtttctatgtcaaaagcaaaacctacgaactctgagtttatcgcaatggtt

SEQ ID NO: 12 - DNA, 280 bp, Artificial Sequence
  Sequence derived from the B_thuringiensis_BMB171 with introduction of three synonymous substitutions.  Codon pairs GGCTCC (83 to 88), GCCTCG (138 to 143) and GTTTCT (230 to 235) replace wild type codon pairs GGATCG, GCAAGC and GTATCC.
aattggtgtacccgctcatattaaaggatatatgtatttacgagaagcaatctccatggtatacaacgatatcgaattattaggctccattacgaaagtattgtatccagatatcgcaaagaaatataatacaacagcctcgcgtgtggagcgcgcaattcgccacgcgattgaagtagcttggagccgtgggaatattgattctatttcgtccttattcggttatacagtttctatgtcaaaagcaaaacctacgaactctgagtttatcgcaatggtt